(19) United States
(12) Patent Application Publication
(10) Pub. No.: US 2016/0081847 A1
(43) Pub. Date: Mar. 24, 2016

(54) 3D-NONWOVEN

(71) Applicant: Gabriele RENNER, Elchingen (DE)

(72) Inventor: Gabriele RENNER, Elchingen (DE)

(21) Appl. No.: 14/863,607

(22) Filed: Sep. 24, 2015

Related U.S. Application Data
(63) Continuation of application No. PCT/EP2014/000550, filed on Mar. 5, 2014.

(30) Foreign Application Priority Data
Mar. 26, 2013 (DE) 10 2013 005 130.4

(51) Int. Cl. A61F 7/02; D04H 3/00; D04H 1/00

(52) U.S. Cl. CPC A61F 7/02; D04H 1/00; D04H 3/00; A61F 2007/0096

(57) ABSTRACT

The invention relates to a three-dimensional nonwoven material which reduces the temperature at specific body parts to a small extent and, as a result, causes a fat-burning action and can therefore reduce fat directly as a result of the removal of energy at said body parts and/or indirectly as a result of specific mechanisms of lipometabolism. In addition, a skin-firming effect can also be caused by the temperature reduction. The invention is characterized in that the body points are defined and the lipolytic, fat-burning and/or skin-firming effect can be reinforced further in the form of the combination of the 3D nonwoven material with lipolytic, fat-burning and/or skin-firming active substances.

The invention relates to a three-dimensional nonwoven material which reduces the temperature at specific body parts to a small extent and thereby brings about a fat-burning effect directly as a result of withdrawing energy from these body parts and/or indirectly through special mechanisms of the fat metabolism, and can thereby reduce fat. Moreover, a skin-firming effect can also be brought about as a result of the temperature reduction. The invention is characterized in that the body areas are defined and the lipolytic, fat-burning or skin-firming effect can be further enhanced in the form of the combination of the 3D-nonwoven material with lipolytic, fat-burning and/or skin-firming active ingredients.

### DESCRIPTION

The human body has a core body temperature of 36.8 degrees Celsius and a temperature distribution of approx. 28-36 degrees Celsius on its body surface. At higher or lower temperatures, the body starts to keep the body in the optimum temperature ranges, in particular at the optimum core body temperature, by means of endogenous thermoregulation since only within this temperature range do all processes within the body function optimally. To do this, the body expends enormous amounts of energy since this process is vital. Even at temperatures of 38° C. or 39° C. (“fever”), the body is weakened and core body temperatures of more than 40° C. can be life-threatening. Similarly, temperature deviations downwards such as e.g. 33-35° C. have significant effects on endogenous processes and can lead to a weakening of the body and health problems.

Reducing skin temperature does not lead to these extreme reactions to the same extent. Nevertheless, in the event of prolonged application of very warm or very cold temperatures to the skin, a reaction can also take place on the body core. However, the skin system can be used, at least for relatively short periods, to withdraw energy from the body without bringing about bodily reactions that are critical to health. Consequently, excessively low temperatures should not be used for the cooling and, if they are, these should be used as far as possible only under supervision.

On account of the energy which is required by endogenous thermoregulation, energy is withdrawn from the body and, as a result, inter alfa fat tissue is broken down in order to supply the necessary energy. Moreover, it has been found in studies that, particularly as a result of local coldness, energy is removed from the body at the cooled place and this leads to a regional breakdown of fat tissue in the body. In particular, so-called brown fat is suitable for communicating at relatively low temperatures of less than 20 degrees Celsius in such a way that fat burning is the result. Research has shown that a lack of brown adipose tissue leads to increased stimulation of the white fat depot, which takes place due to messengers of the sympathic nerve system. It has been found that the brown adipose tissue communicates with the white adipose tissue in order to regulate body temperature. Conversely, by activating brown adipose tissue through cooling, the breakdown of white adipose tissue can be forced.

Consequently, by cooling the skin, central and regional effects on fat breakdown or fat burning can be brought about, which are also referred to in this document as lipolytic effects.

In this connection, it has been found that the temperature reduction on the skin requires only a slight cooling of approx. 24-28° C. skin temperature and cooling with a material which provides 15-20° C. is sufficient. However, each person reacts somewhat differently to temperatures, meaning that individual adaptation and flexibility of the temperature setting is sensible.

Since the increase in obesity and subsequent diseases such as e.g. diabetes and cardiovascular diseases has become not only a health problem for the individual, but, due to the enormous increase in this problem, also an enormous burden on the healthcare system, it makes sense to make progress with new methods of degrading fat in the body alongside known measures. Moreover, it is known that even in otherwise slim people, the risk of heart attack is increased with increased stomach fat, meaning that this risk factor too could be minimized. Furthermore, beauty aspects, which want to see fat depots at unwanted places reduced, can also profit here, ranging to types of sport such as bodybuilding in which any fat depot that adversely affects the defined view of musculature is troublesome.

These methods must be able to be carried out primarily for the groups of people mentioned first in a quick and simple manner and at best while mobile and by those affected themselves, including at home. The application must, moreover, function without great effort or side effects and be pleasant.

As one knows from the studies on diets or exercise activities, success is related significantly to the acceptance by the user. An unpleasant, costly, time-intensive or unpleasant measure will be carried out either not at all or not consistently and therefore also does not lead to success. Furthermore, these diseases are a “people's disease”, as a result of which the costs for any measures must not be too high since these can otherwise not be borne by health insurance companies or by the health system.

A number of cooling methods are already known in the prior art, but these have some disadvantages:

One very complex method involves cooling chambers in which one dwells for a few minutes in rooms or large containers cooled to below 100-to 150° C. However, these cooling chambers do not exist extensively on account of the cost and the energy and space requirement. Moreover, these minus temperatures are very unpleasant and must be carried out strictly under supervision. Studies relating to these cooling rooms and their effect on fat reduction, however, are hitherto unavailable.

Furthermore, there are apparatuses which squeeze, the area to be cooled which has the fat depots, between plates (pressure build-up) and these plates are moreover cooled by this apparatus. As a result of the pressure and the cooling, according to this arrangement the fat cells are made to split and the destroyed adipose tissue is transported away in the following 4-8 weeks. However, the application must be carried out by personnel trained for it with supervision. The application is not mobile, meaning that the time for this must be found and not all body parts can be inserted in this form between the plates, meaning that the method is not a whole-body method and can thus only be used for some body parts. Moreover, corresponding costs arise as a result of using the very complex application, the energy source and the personnel.

A further product for cooling is a cotton suit consisting of a very light thin cotton fabric similar to a gauze bandage material which is impregnated in a liquid of alcohol, caffeine, carnitine and coenzyme A.

This suit is then worn and is intended to serve for fat reduction. The suit is naturally wet and unpleasant due to the liquid, there is a risk of getting a cold due to the wet-cold humidity and, moreover, the material cannot be washed and/or be reused for an extended time. This makes application over an extended time expensive.

A further cooling arrangement in the prior art is given by a PCM system (PCM=phase change material), in which salt, which melts at 14° C., is used, which is incorporated into vests heat-sealed in plastic. These salts melt and, in so doing, give off a cooling effect, which is said to serve for fat reduction. Since the time of melting, at approx. 60-90 min, is limited, as a result of storage and regeneration in the refrigerator or freezer, correspondingly complex logistics are required, with corresponding costs. The vest has a considerable weight as a result of using the salt pack—moreover, the cooling temperature of long-lasting 14° C. is relatively cold and is not tolerated by every user. The salt packs in the vest are not only heavy, but also hard on the skin and not smooth. Moreover, water of condensation is formed as a result of the cooling and this will make the user wet and damp.

Consequently, all of the cooling arrangements available hitherto have clear disadvantages. The aim of the present invention is to overcome the disadvantages of the arrangements hitherto and to provide a rapid, simple, dry cooling arrangement with the aim of high acceptance by the user and low cost structure.

The present invention proposes a three-dimensional nonwoven material which, in order to utilize the advantages of the surface expansion, is at least 2 mm in thickness, can absorb liquids, and binds these firmly, so that no liquid touches the skin in the form of liquid drops and the body remains dry as a result. The nonwoven material has a three-dimensional irregular network structure which has superabsorbers on or in the nonwoven fibers interconnected like a network. The liquid binding takes place at the fibers and their linkage points, which increases the binding of the liquid and enlarges the surface for the binding of the liquid. As a result, the liquid is firmly bonded and cannot escape as a result of pressure. The surface is therefore dry and the body does not become wet during use. By using a textile or nontextile covering around the nonwoven material, which is liquid-permeable and liquid-vapor-permeable either entirely or at least at the top of the side pointing away from the body, a corresponding design of the invention can also be made possible in order to increase the acceptance of the user. The material is washable and therefore can be used again and again, which saves costs.

This increases the compliance of the method compared to the prior art, which uses a two-dimensional cotton material with a gauze-bandage-like structure which is wet and drips, and significantly reduces the possible side effects which could arise due to the wet material. The reusability is likewise an advantage over the prior art since although the gauze-bandage-like material can be used several times, it is not washable and then must be disposed of for hygiene reasons and a new product has to be used, which again produces costs.

Cooling in the three-dimensional nonwoven material according to the invention is ensured by evaporation of the liquid, which arises on account of the three-dimensional nature and consequently large surface of the liquid present in the nonwoven material, which releases the liquid again to a great extent and thereby produces temperatures of 15-20° C. which, when used on the skin, leads to skin temperatures of 24-28° C. and thereby brings about a lipolytic and thus fat-reducing effect.

By adding lipolytically acting substances to the material, the absorbed liquid or separately by using a cosmetic, the effect is synergistically enhanced. For this purpose, besides plant active ingredients and preparations, it is also possible to use isolated active ingredients as well as active ingredients produced by chemical or biotechnological means. Here, active ingredients can be used individually, or in combination or in mixtures. These active ingredients can be used in the form of a liquid, a gel, a cream or another medicinal, pharmaceutical or cosmetic preparation. Besides the lipolytically acting substances, it is also possible to use active ingredients which play a promoting role in fat metabolism. The effects on skin firming found during investigations for the invention, the antiwrinkle effect and the anticellulite effect, an addition of skin-firming, antiwrinkle and/or anticellulite active ingredients is also sensible.

Moreover, active ingredients can be used which increase the penetration of active ingredients through the skin in order to increase the concentration of the active ingredients at the site of action. This is useful since the absorption of active ingredients becomes more difficult due to the cooling, possibly e.g. as a result of vasoconstriction.

Such substances such as e.g. alcohols or alcoholic mixtures or solutions, salicylic acid, DMSO, other sulfoxides or urea and further substances which improve the absorption of active ingredients into the skin (absorption accelerators) can be used here.

Furthermore, fragrances such as e.g. essential oils or other fragrances, as well as skin care active ingredients such as e.g., but not exclusively, aloe vera, dexpanthenol, calendula, zinc, etc. can be used in order, on the one hand, in the case of the fragrances, to promote the compliance, i.e. the acceptance for the user, and secondly to provide a skin care effect in order to reduce any skin damage which could arise on sensitive skin as a result of the cooling.

The aim is to offer a rapid, simple, mobile and pleasant cooling arrangement which can be used again and again gladly and even organizatorily by the people affected.

In order to permit control of fat reduction both of the cooling effect and also of the lipolytic effects, this control was co-developed for the object according to the invention.

In order to ensure the control of cooling efficiency, the cooling temperature is measured. This can take place in the form of an integrated thermometer, a device with thermocolors or thermocrystals or other known temperature measurement devices.

In order to see the control of the weight loss, measuring devices in the form of balances, fat measuring instruments, girth measuring instruments, etc. are used either on the cooling system or separately but in combination therewith, which reveals, as a result of automatic transfer or through input, the efficiency of the cooling device in the form of a documented result. This may be a monitor or may be a PC, tablet, mobile telephone, mp3 player, or other stationary or mobile terminal. The results can either be sent for self-monitoring or else, by means of transmission to a third party, for monitoring by a doctor, nutritional adviser or to a platform on the Internet which performs an evaluation and offers feedback with further suggestions for improvement in order to increase the efficiency. As a result of this, the acceptance and thus compliance can be improved with appropriate reports of success and motivational elements. This can also be performed, by way of play, with Apps or other programs in the stationary or mobile terminal, Intranet or Internet or other communication devices.

The aim is to support those affected during fat reduction and to increase compliance. As numerous studies show, the acceptance and perseverance with the measures for fat reduction are the most important factor for success.

Only then does a person affected stick to it and achieve long-term successes which serve their health and take the strain off the health system by avoiding subsequent diseases.

The cooling arrangement can of course also be used in combination with sport, mobility and/or nutrition adjustment or diets in order to achieve a more rapid success through synergistic effects of the individual methods. Precisely in this respect, mobile use, including at home, is advantageous.

Here too, a combination of all of these methods via new media is sensible and is supported by the cooling arrangement according to the invention.

Furthermore of importance is the arrangement of the cooling nonwoven on the body by so-called body mapping in which the regions are ascertained which are to be cooled. Here, it is important to omit areas which should not lose volume, such as e.g. the breast area in women. The same is true for areas that are sensitive to cold such as e.g. the area around the kidneys and the urinary tract and possibly the genitals.

Moreover, in a further embodiment of the invention, it is useful to bind the areas into the cooling ranges which are notable for the fact that they are areas which include brown fat in order, besides the energy benefit, to enhance the communication of fat metabolism and to thereby ensure additional effects on fat reduction. These include, besides the shoulder blades, the neck and the spinal column.

Compression of the affected body parts by elastic, narrow substances compressing the body parts can likewise bring about a positive effect. One embodiment according to the invention would also be a model in the form of shaping underwear which includes the nonwoven material according to the invention, in a fixed or detachable manner, on defined areas or places on the body. However, wraps and compression clothing or compressive or simple bandages on certain body parts are also provided by the invention.

The articles equipped with the nonwoven material according to the invention over all or part of their area can correspond to different body shapes or only be intended for certain places on the body. Thus, it is possible to equip clothing such as tops with or without sleeves, short- or long-sleeved, vests, shirts, jackets, dresses, suits, overalls, shaping hosiery and clothing, compression clothing or accessories, skirts, underwear, trousers, leggings, chaps, stockings, clothing constituents, masks, eye masks, wraps, dressings or bandages, pads for face and/or other body parts. Wipes, cloths or other variously shaped coverings for people and/or animals can be used, or the nonwoven material can be incorporated in the form of pads, in a fixed or detachable manner, in or on clothing or clothing constituents, wraps, dressings or bandages, cloths, masks or eye masks.

In this connection, the nonwoven according to the invention can be integrated into the specified articles in a fixed or detachable manner. Pads which contain the nonwoven material and are joined with closures or incorporated into pockets can be used, or the nonwoven material can be sewn in, heat-sealed in, stitched in or firmly attached in another manner into the article over the whole area or at certain places.

In our investigations, it has been found that by cooling the corresponding body parts when using the cooling arrangement daily or every other day, e.g. a girth reduction in the stomach area of 2-4 cm was achieved within two or four weeks, which corresponds to half to one dress size. In individual cases, in four weeks and longer, a girth reduction of 4-8 mm was also found, which corresponds to a reduction of two dress sizes. Precisely the different action mechanisms of the regional cooling are evident in the results.

Table Results:

Stomach circumference upon daily cooling of at least 30 min using the 3D nonwoven, water and in combination with 5% caffeine and cooling daily or every two days.

| | Measured stomach girth in cm | | |
|---|---|---|---|
| Time | Subject A | Subject B | Subject C |
| Day 1 | 89 | 91 | 101 |
| Day 2 | 89 | | 101 |
| Day 3 | 88 | 91 | 100.5 |
| Day 4 | 88 | | 100.5 |
| Day 5 | 87.5 | 90.5 | 100.5 |
| Day 6 | 87.5 | 90.5 | 100.5 |
| Day 7 | 87 | | |
| Day 8 | | 91 | 100 |
| Day 9 | 87 | | 100 |
| Day 10 | 87 | 90.5 | 100 |
| Day 11 | 87 | | |
| Day 12 | | 90.5 | 99.5 |
| Day 13 | 86.5 | 90 | 99.5 |
| Day 14 | 86.5 | 90 | 99.5 |
| Day 15 | 86.5 | 90 | 99.5 |
| Day 16 | | | 99 |
| Day 17 | 86 | 89.5 | 99 |
| Day 18 | | 89.5 | 99 |
| Day 19 | 86.5 | | 99 |
| Day 20 | | 90 | |
| Day 21 | 86 | | 99.5 |
| Day 22 | | 89.5 | 99.5 |
| Day 23 | 86 | 89.5 | 99 |
| Day 24 | | | |
| Day 25 | 85.5 | 89 | 99 |
| Day 26 | 85.5 | 89 | 98.5 |
| Day 27 | 85 | 88.5 | 98.5 |
| Day 28 | 85 | 88.5 | 98.5 |

Moreover, it was found in the studies carried out that the cooling also had positive effects on the skin structure and the connective tissue, which was evident from a reduction in wrinkles, a more uniform skin structure and reduced cellulite. Since the skin structure becomes smoother as a result of the reduced fat depots, it is also demonstrated that, as a result of cold, e.g. circulation is stimulated and collagen production is increased. Consequently, besides skin-firming effects, it is also possible to achieve wrinkle-reducing effects. The skin-firming effect can thus also have a positive effect to combat the appearances of cellulite.

In one embodiment according to the invention, the use of the cooling nonwoven is such that the cooling nonwoven is incorporated with a water-permeable covering into an upper clothing garment and/or a lower clothing garment which lies against the body. This can take place by directly incorporating the cooling nonwoven into the item of clothing or by means of pads which are attached in pockets or in some other way into or onto the item of clothing.

The cooling nonwoven is impregnated, poured over or sprayed with a liquid such as e.g. water either with the item of clothing or the cooling nonwoven pads before attachment or incorporation into the item of clothing, such that approx. 1 ml of liquid is contained per cm² in the nonwoven. If more liquid is used, this is also bonded to a high degree. The product will only become heavier as a result, but the function is retained. If too much liquid is used, the cooling nonwoven can simply be lightly squeezed mechanically following the addition of liquid and, if necessary, the covering can be dried by drying off using a cloth or by other methods such that water no longer escapes in the form of drops from the cooling nonwoven. Should a covering be used which does not ensure rapid drying, the article can be dried superficially using heat (heating, hairdryer, or the like). The liquid nevertheless remains firmly bonded to more than 90% in the cooling nonwoven and cannot escape further as a result of pressure. In the device according to the invention, the article is then ready-to-use and can be used for cooling. The body itself does not become wet due to the contained liquid, which contrasts the present invention significantly from the prior art and therefore leads to increased acceptance by the user. As a result of the binding of the liquid in the three-dimensional network structure of the nonwoven, the surface is very enlarged and therefore the liquid surface is also very enlarged, which leads to an increased evaporation capacity and thus to greater cooling than in the case of a simple two-dimensional material structure. Moreover, this increased surface absorbs liquid better and more quickly and binds it as a result and, as a result of the superabsorbers bonded in the three-dimensional nonwoven structure on the crosslinked fibers, the liquid becomes firmly bonded, meaning that it does not escape due to pressure and therefore keeps the body dry. This improves the wear properties enormously compared to the prior art and offers clear advantages during application.

The other active ingredients which are used synergistically for the cooling in order to further enhance the effect are either incorporated into the nonwoven in the form of admixing with the liquid, or the nonwoven and/or the covering is equipped with such active ingredients and/or the active ingredients are applied to and/or introduced onto the skin in one of the described preparations before using the cooling fleece. Besides the described preparations in the form of liquids, sera, gels, creams or other topical or dermal preparations, this can also through transdermal systems, patches, micro-needles and similar systems. Injections with or without needles, or other systems in the prior art can naturally likewise be used.

Directly after application or incorporation of the active ingredients to or into the skin, if used in this form and not already sufficiently integrated in the nonwoven or in the covering, the article in which the cooling nonwoven with corresponding covering is firmly or detachably integrated is correspondingly worn by the user or placed onto the skin.

After approx. 5-10 minutes, the article is briefly removed and aerated and worn again or applied again with the hitherto outer side facing inwards towards the skin. This swapping achieves a higher cooling effect since, as a result, the heat of the body present is regionally absorbed by the cooling nonwoven and transported away. The other side of the cooling nonwoven is significantly cooler and can withdraw further heat from the tissue, as a result of which the target temperature of 24-29° C. on the skin is achieved rapidly and continuously. This operation is repeated as soon as the cooling nonwoven on the side facing towards the skin warms up again to a significant extent.

This thus always takes place when the temperature on the inside or on the skin warms up. As a result of this process, energy is removed from the skin and/or the body and the fat-reduction process is set in motion.

In those affected for whom the cooling effect on its own due to evaporative coldness does not suffice, the effect can be enhanced by precooling the material in the refrigerator, in a coolbox or another cooling container or by incorporating standard commercial ice packs or frozen gel packs or active PCM pads onto the outside of the nonwoven. The ice, gel or PCM material here should not come into direct contact with the skin in order to prevent the discussed negative effects. In no case should the precooling lead to temperatures lower than 10° C. in order to prevent health damage due to cooling on the skin and/or the body (such as e.g. to the immune system). The three-dimensional nonwoven material rapidly absorbs the coldness from the externally attached ice or gel or PCM pack on account of its large surface and transmits this in attenuated form to the skin. Through insertion possibilities into pockets or detachably applied e.g. with Velcro fastening, these cooling enhancers can also only be used in certain places or be removed or detached again after a certain time.

A brief firm massage of the affected body parts directly after the cooling has also proven to be advantageous since the shattered fat cells are further destroyed through the mechanical pressure the through the cooling and, if used, the lipolytically effective active ingredients. The rate at which these cells are transported away is increased. Lymph drainage or methods corresponding to this also additionally facilitate the transporting away of the fat cells.

In this connection, reference is made in particular to the fact that all of the features and properties described with regard to the three-dimensional cooling nonwoven and its textile or nontextile covering or the cooling arrangement and the cooling method, but also procedures can also be applied analogously with regard to the wording of the process according to the invention and can be used in the context of the invention and are deemed to be co-disclosed. The same is also true vice versa. This means that structural, thus material features specified only in relation to the process or the method or the cooling arrangement can also be claimed within the scope of the claims for the three-dimensional nonwoven, the cooling arrangement or the textile or nontextile covering, as well as the medicinal, pharmaceutical and/or cosmetically effective substances used, and likewise form part of the invention and the disclosure.

### DESCRIPTION OF THE VIEWS IN THE DRAWING

The invention is shown in diagrammatic form in the drawing.

FIG. 1 shows

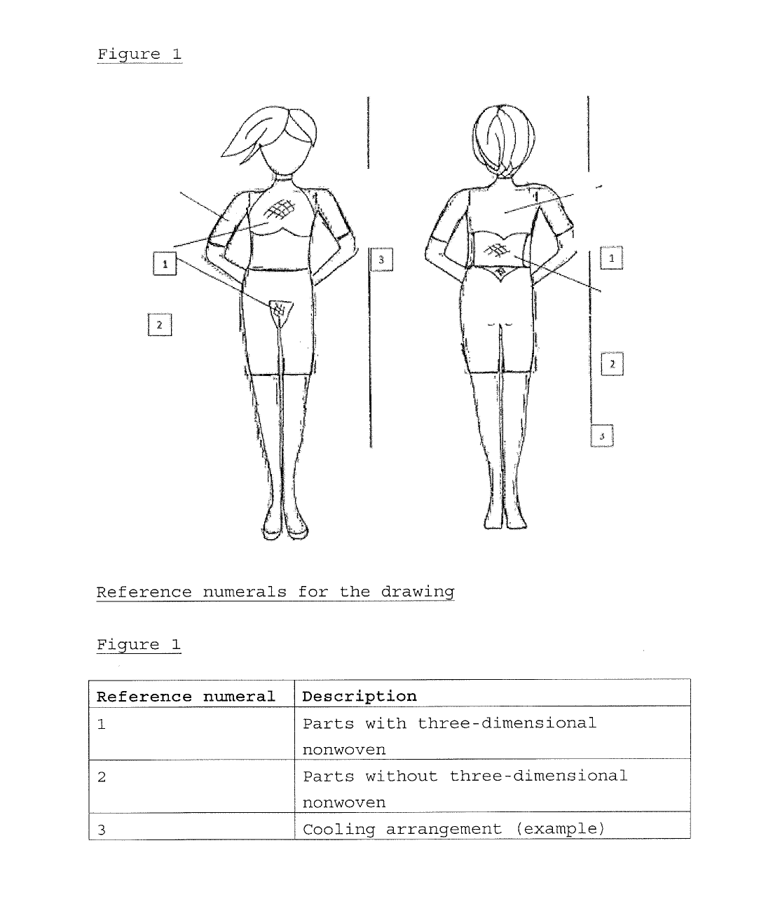

A front and back view of a variant of a cooling arrangement (3) according to the invention in the form of an item of clothing consisting of parts with a three-dimensional cooling nonwoven (1) and with parts without a three-dimensional cooling nonwoven (2), since these areas are not to be cooled in the sense of body mapping since these areas are either particularly sensitive to cold, such as e.g. the kidneys or areas of the urinary tract and/or genitals, or these areas are not to be cooled since a reduction in volume in these areas is undesired such as e.g. in the breast area.

The claims submitted now with the application and subsequently are attempts at wording without prejudice for achieving extensive protection. The working examples of the invention explained above serve merely for the purpose of better understanding of the teaching according to the invention defined by the claims which are per se not limited by the working examples. Should closer examination, particularly of the relevant prior art, reveal that one or other feature, albeit favorable, is not decisively important for the aim of the invention, then naturally a wording will already be aimed at which no longer has such a feature, especially in the main claim.

It should also be taken into consideration that the configurations and variants of the invention described in the various embodiments and shown in the figures can be combined with one another as desired. In this connection, some or all of the features can be exchanged for one another as desired. These feature combinations are likewise co-disclosed.

The back-references listed in the dependent claims point to the further development of the subject matter of the main claim by virtue of the features of the respective dependent claim. However, these are not to be understood as being dispensation for achieving independent, material protection for the features of the dependent claims to which a back-reference is made. Features which have hitherto only be disclosed in the description may be claimed in the course of the proceedings as being of essential importance to the invention, for example for the purposes of delimitation from the prior art.

Features which have only been disclosed in the description, or else individual features from claims which include several features can be transferred at any time to the first claim for the purposes of delimitation from the prior art, specifically if such features have been mentioned in connection with other features, or attain particularly favorable results in connection with other features.

1. A three-dimensional nonwoven material surrounded with textile or nontextile material and having a thickness of 2 mm-10 mm which, in conjunction with liquids incorporated therein, cools body areas and has a fat-burning action.

2. The three-dimensional nonwoven material surrounded with textile or nontextile material as claimed in claim 1, characterized in that, as a result of the cooling effect on certain body parts, fat is broken down on the same body parts or on different body parts as a result of the fat metabolism.

3. The three-dimensional nonwoven material surrounded with textile or nontextile material as claimed in claim 1, characterized in that its fibers and/or structures are coated with superabsorbers or are firmly attached to superabsorbers in another way and/or are formed from superabsorbent fibers which form a three-dimensional network structure.

4. The three-dimensional nonwoven material surrounded with textile or nontextile material as claimed in claim 1, characterized in that the nonwoven material surrounded with textile or nontextile material, after introducing liquid and squeezing out the nonabsorbed liquid and/or by drying the outer surface, feels dry and/or no longer releases liquid in the form of drops and keeps the liquid in bonded form and only releases it again as a result of evaporation.

5. The three-dimensional nonwoven material surrounded with textile or nontextile material as claimed in claim 1, characterized in that it produces a skin-appearance-improving or skin-firming and/or wrinkle-diminishing and/or anticellulite effect.

6. The three-dimensional nonwoven material surrounded with textile or nontextile material as claimed in claim 1, characterized in that it is used in synergistic effect with lipolytic and/or skin-firming and/or wrinkle-diminishing and/or anticellulite substances.

7. The three-dimensional nonwoven material surrounded with textile or nontextile material as claimed in claim 1, characterized in that it is used together with medicinal and/or pharmaceutical and/or cosmetic active ingredients which have a lipolytic and/or wrinkle-diminishing and/or skin-firming and/or anticellulite effect, and these aforementioned active ingredients are already firmly or detachably incorporated in and/or applied to the nonwoven material and/or these active ingredients are applied or incorporated in a preparation with the liquid used as claimed in claim 4 and/or these active ingredients are applied to the skin beforehand before applying the nonwoven to the skin for the purpose of cooling.

8. The three-dimensional nonwoven material surrounded with textile or nontextile material as claimed in claim 1, characterized in that the nonwoven material is surrounded, for protection and for reasons of handleability and also of design, with a covering which is formed of textile or nontextile materials and this arrangement is used for cooling.

9. A textile and/or nontextile covering of the nonwoven material as claimed in claim 1, characterized in that it is equipped and/or coated with lipolytic and/or wrinkle-diminishing and/or skin-firming and/or anticellulite substances and/or has a reservoir of active ingredients that are lipolytic and/or wrinkle-diminishing and/or skin-firming and/or anticellulite or favor fat-burning.

10. The textile and/or nontextile covering of the nonwoven material as claimed in claim 9, characterized in that this is liquid-permeable and/or is liquid-permeable in at least one place and/or in or on which there is an option of incorporating liquids and which permits the evaporation of one or more liquid constituents or of the entire liquid from the three-dimensional nonwoven.

11. A cooling arrangement as claimed in claim 1, characterized in that the three-dimensional nonwoven material surrounded with textile or nontextile material is used in the form of clothing such as tops with or without sleeves, short- or long-sleeved, vests, shirts, jackets, dresses, suits, overalls, shaping hosiery and clothing, compression clothing or accessories, skirts, underwear, trousers, leggings, chaps, stockings, clothing constituents, masks, eye masks, wrappings, dressings or bandages, pads for face and/or other body parts, cloths, or of other variously shaped coverings for people and/or animals, or is used in the form of pads firmly or detachably in or on the specified clothing or clothing constituents, wraps, dressings or bandages, cloths, masks or eye masks and cools as a result of evaporative coldness and/or only parts of the specified forms consist of the three-dimensional nonwoven material surrounded with textile and/or nontextile material or include pads thereof and other parts (2) of said forms consist only of textile or nontextile material and characterized in that the cooling arrangement is washable and can be reused many times.

12. The cooling arrangement as claimed in claim 11, characterized in that it is used together with medicinal and/or pharmaceutical and/or cosmetic active ingredients which have a lipolytic and/or wrinkle-diminishing and/or skin-firming and/or anticellulite effect and these aforementioned active ingredients are already firmly or detachably incorporated in and/or applied to constituents of the cooling arrangement and/or these active ingredients are applied or incorporated in a preparation with the liquid used and/or these active ingredients are incorporated in or applied to the skin before applying the cooling arrangement to the skin.

13. The cooling arrangement as claimed in claim 1, characterized in that it is precooled in a cooling container and/or by applying colder substances or materials to the side of the cooling nonwoven facing outwards and this leads to a cooling intensification.

14. The cooling arrangement as claimed in claim 1, characterized in that, to control the cooling efficiency, measuring instruments such as thermometers and/or other measurement methods suitable for measuring temperature and/or measuring instruments for measuring weight and/or fat and/or girth measurement and/or measurement of other body parameters are combined and/or that this is used for monitoring the effectiveness and effect by the user himself or by third parties or in communities with the help of stationary or mobile IT systems or communication systems, programs and/or software and hardware.

15. A lipolytic, fat-burning and/or skin-firming and/or anticellulite cooling method which involves the following parameters:
provision of a three-dimensional nonwoven material surrounded with textile or nontextile material as claimed in claim 1,
use of medicinal, pharmaceutical or cosmetically active substances which have a lipolytic and/or skin-firming and/or anticellulite effect and which are applied or incorporated directly in or on the skin or which are firmly or detachably attached in or to the nonwoven material and/or in or to the textile or nontextile material
incorporation of liquid into the three-dimensional nonwoven material surrounded with textile or nontextile material,
attachment or application of the dry, liquid-laden three-dimensional nonwoven material surrounded with textile or nontextile material to the skin of a body,
evaporation of the liquid and/or of liquid constituents for the purpose of cooling the skin and the fat layers underneath.
